(19) United States
(12) Patent Application Publication
(10) Pub. No.: US 2025/0194974 A1
(43) Pub. Date: Jun. 19, 2025

(54) BUTTERFLY NEEDLE ADAPTER DEVICE

(71) Applicant: AIRS MEDICAL INC., Seoul (KR)

(72) Inventors: Baesang JANG, Gyeonggi-do (KR); Yeonbo SHIM, Seoul (KR); Dongmyung MIN, Gyeonggi-do (KR)

(21) Appl. No.: 19/069,228

(22) Filed: Mar. 4, 2025

Related U.S. Application Data
(63) Continuation of application No. PCT/KR2023/009481, filed on Jul. 5, 2023.

(30) Foreign Application Priority Data
Sep. 5, 2022 (KR) 10-2022-0111876

(51) Int. Cl. A61B 5/15

(52) U.S. Cl. CPC A61B 5/150732; A61B 5/15074

(57) ABSTRACT

The butterfly needle adapter device may include a second clamp configured such that the butterfly needle is inserted and seated thereinto and a first clamp configured to prevent the butterfly needle from being separated from the second clamp in a hold mode, and the second clamp may include a separation guide portion that guides the butterfly needle through separation via one side of the second clamp in a release mode.

### CROSS-REFERENCE TO RELATED APPLICATION

This application is a Bypass Continuation Application of PCT/KR2023/009481 filed on Jul. 5, 2023, which claims priority to Korean Patent Application No. 10-2022-0111876 filed on Sep. 5, 2022. The entire disclosure of each of the foregoing applications is incorporated herein by reference in its entirety.

### TECHNICAL FIELD

The present disclosure relates to a butterfly needle adapter device that enables a butterfly needle to be easily inserted and separated in order to efficiently perform automatic blood collection.

### BACKGROUND ART

In general, blood collection refers to the collection of blood required for clinical pathology experiments or various tests from blood vessels of blood collection targets.

There are various blood collection methods depending on the purpose of blood collection. Generally, when only a small amount of blood needs to be collected for blood type testing or simple disease diagnosis, a scalpel, a lancet, or a Franke needle is used to collect blood by slightly tearing the skin on the earlobe, fingertip, or sole of the foot.

In contrast, when a large amount of blood needs to be collected, blood is collected by puncturing the blood vessel of a blood collection target with a needle and then forming negative pressure inside the needle by means of a blood collection syringe.

This type of blood collection requires a needle to be accurately inserted into a blood vessel of a blood collection target, and thus, it is important to find the blood vessel of the blood collection target. When the blood vessel of the blood collection target is not clearly visible, a problem arises in that it is difficult to find the blood vessel, making blood collection difficult.

In order to overcome this problem, there has been developed a blood collection device that detects the blood vessel of a blood collection target, automatically punctures the blood vessel of the blood collection target with a needle part, and then collects blood. This has brought about various changes in the health and medical field.

In this blood collection device, blood is collected using a butterfly needle that is detachably fixed to a butterfly needle adapter device.

However, the conventional butterfly needle adapter device has a problem in that it is inconvenient to fix and separate the butterfly needle.

In addition, the conventional butterfly needle adapter device has a problem in that there is a considerably high possibility that a user is pricked by the needle part of the butterfly needle during the process of fixing and separating the butterfly needle.

### DISCLOSURE

### Technical Problem

The present disclosure has been conceived in response to the above-described background art, and a butterfly needle adapter device according to one embodiment of the present disclosure is intended to enable a butterfly needle to be easily inserted and separated into and from the butterfly needle adapter device in order to efficiently perform automatic blood collection.

In addition, a butterfly needle adapter device according to one embodiment of the present disclosure is intended to include a separation guide portion, thereby intuitively and easily performing the separation or removal of a butterfly needle.

In addition, a butterfly needle adapter device according to one embodiment of the present disclosure is intended to include a separation guide portion so that a butterfly needle is separated via a side of the butterfly needle adapter device after blood collection, thereby preventing the butterfly needle adapter device from being contaminated with contaminants attached thereto when the butterfly needle is separated via the rear side of the butterfly needle adapter device and also preventing secondary infection.

In addition, a butterfly needle adapter device according to one embodiment of the present disclosure is intended to include a hold guide portion, thereby enabling the butterfly needle to be fixed without moving during blood collection.

In addition, a butterfly needle adapter device according to one embodiment of the present disclosure is intended to include an insertion guide portion, thereby enabling a butterfly needle to be easily inserted from the rear end of a first clamp and a second clamp to the front end thereof.

However, the objects to be accomplished by the present disclosure are not limited to the objects mentioned above, and other objects not mentioned will be clearly understood based on the following description.

### Technical Solution

There is disclosed a butterfly needle adapter device according to one embodiment of the present disclosure for achieving the above-described object. The butterfly needle adapter device may include a second clamp configured such that the butterfly needle is inserted and seated thereinto and a first clamp configured to prevent the butterfly needle from being separated from the second clamp in a hold mode, and the second clamp may include a separation guide portion that guides the butterfly needle through separation via one side of the second clamp in a release mode.

In the present disclosure, there may be provided the butterfly needle adapter device, wherein in an insert mode, the butterfly needle is inserted from the rear end side of the second clamp to the front end side thereof.

In the present disclosure, there may be provided the butterfly needle adapter device, wherein the separation guide portion includes a separation guide recess that is disposed at the front end of the second clamp and formed to extend toward one side of the second clamp.

In the present disclosure, there may be provided the butterfly needle adapter device, wherein the separation guide portion includes a separation inclined portion that extends on one side of the separation guide recess and is inclined downward to one side.

In the present disclosure, there may be provided the butterfly needle adapter device, wherein the second clamp includes a butterfly needle hold guide portion at the front end thereof and the hold guide portion includes a first protrusion protruding convexly downward.

In the present disclosure, there may be provided the butterfly needle adapter device, wherein the hold guide portion includes a second protrusion that extends in one lateral direction and protrudes convexly downward.

In the present disclosure, there may be provided the butterfly needle adapter device, wherein the second clamp includes an insertion guide portion that guides the butterfly needle through insertion in an insert mode.

In the present disclosure, there may be provided the butterfly needle adapter device, wherein the insertion guide portion includes a first inclined guide portion that is inclined downward from the rear end side of the second clamp toward the front end side thereof.

In the present disclosure, there may be provided the butterfly needle adapter device, wherein the insertion guide portion includes a second inclined guide portion that is inclined downward from the rear end side of the second clamp toward the front end side thereof and is also inclined upward in the one lateral direction.

In the present disclosure, there may be provided the butterfly needle adapter device, wherein the insertion guide portion includes a straight movement guide portion that extends straight from the rear end side of the second clamp toward the front end side thereof.

In the present disclosure, there may be provided the butterfly needle adapter device, wherein the first clamp includes a seat depression in which the butterfly needle is seated in the hold mode.

In the present disclosure, there may be provided the butterfly needle adapter device, wherein the seat depression is open forward of the first clamp.

In the present disclosure, there may be provided the butterfly needle adapter device, wherein the rear end of the seat depression is closed and thus prevents the butterfly needle from retreating in the hold mode.

In the present disclosure, there may be provided the butterfly needle adapter device, wherein the first clamp includes a reception portion into which the first protrusion is inserted in the hold mode.

### Advantageous Effects

The butterfly needle adapter device according to one embodiment of the present disclosure enables the butterfly needle to be easily inserted and separated into and from the butterfly needle adapter device in order to efficiently perform automatic blood collection.

In addition, the butterfly needle adapter device according to one embodiment of the present disclosure includes the separation guide portion, thereby intuitively and easily performing the separation or removal of the butterfly needle.

In addition, the butterfly needle adapter device according to one embodiment of the present disclosure includes the separation guide portion so that the butterfly needle is separated via a side of the butterfly needle adapter device after blood collection, thereby preventing the butterfly needle adapter device from being contaminated with contaminants attached thereto when the butterfly needle is separated via the rear side of the butterfly needle adapter device and also preventing secondary infection.

In addition, the butterfly needle adapter device according to one embodiment of the present disclosure includes the hold guide portion, thereby enabling the butterfly needle to be fixed without moving during blood collection.

In addition, the butterfly needle adapter device according to one embodiment of the present disclosure includes the insertion guide portion, thereby enabling the butterfly needle to be easily inserted from the rear end of the first clamp and the second clamp to the front end thereof.

### DESCRIPTION OF DRAWINGS

FIG. 1 is a perspective view showing a butterfly needle according to one embodiment of the present disclosure;

FIG. 2 is a perspective view showing a butterfly needle adapter device and the hold mode of a butterfly needle according to one embodiment of the present disclosure;

FIG. 3 is a diagram showing a first clamp according to one embodiment of the present disclosure;

FIG. 4 is a diagram showing a second clamp according to one embodiment of the present disclosure;

FIG. 5 is a diagram showing the front end of a butterfly needle adapter device according to one embodiment of the present disclosure; FIG. 6 is a diagram showing a butterfly needle adapter device including a driving unit according to one embodiment of the present disclosure;

FIG. 7 is a diagram illustrating the operation of the driving unit in a hold mode according to one embodiment of the present disclosure;

FIG. 8 is a diagram illustrating the operation of the driving unit in a release mode according to one embodiment of the present disclosure;

FIG. 9 is a diagram showing an insert mode for a butterfly needle according to one embodiment of the present disclosure; and

FIG. 10 is a diagram showing a release mode for a butterfly needle according to one embodiment of the present disclosure.

### MODE FOR INVENTION

Embodiments of the present disclosure will be described in detail below with reference to the accompanying drawings so that those having ordinary skill in the art of the present disclosure (hereinafter referred to as those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings. The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “at least one of A and B” used herein should be interpreted to refer to all of A, B, and combinations of A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, it should be interpreted that a singular form may generally include “one or more.”

The term “N-th (N is a natural number)” used herein may be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “connected” used therein should be interpreted to include not only a case where components are “directly connected” to each other but also a case where a third component is “present” between components and a case where components are “electrically connected” to each other with another component disposed therebetween.

The foregoing descriptions of the terms are intended to help understanding of the present disclosure. Accordingly, it should be noted that unless the above-described terms are explicitly described as limiting the content of the present disclosure, they are not used in the sense of limiting the technical spirit of the present disclosure.

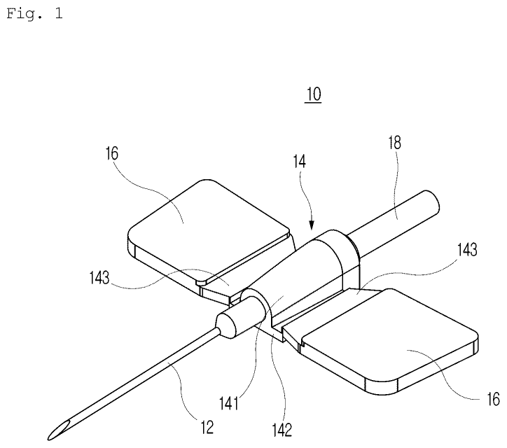

FIG. 1 is a perspective view showing a butterfly needle according to one embodiment of the present disclosure.

Referring to FIG. 1, a butterfly needle 10 may include a needle part 12, a needle support part 14 configured to support the needle part 12, a pair of wings 16 disposed opposite each other with the needle support part 14 interposed therebetween, and a tube 18 configured to communicate with the needle part 12 via the needle support part 14.

The needle support part 14 may include a needle connection support portion 141 including a cylindrically curved portion whose diameter decreases from the rear end thereof to the front end thereof, plate-shaped base support portions 142 supporting the cylindrically curved portion on the bottom thereof, and wing support portions 143 protruding upward from the base support portions 142 and having side ends connected to the wings. The wings 16 may be made of a flexible material having elasticity. The tube 18 may have a diameter relatively smaller than that of the needle support part 14. Furthermore, the needle part 12 may have a diameter relatively smaller than that of the tube 18.

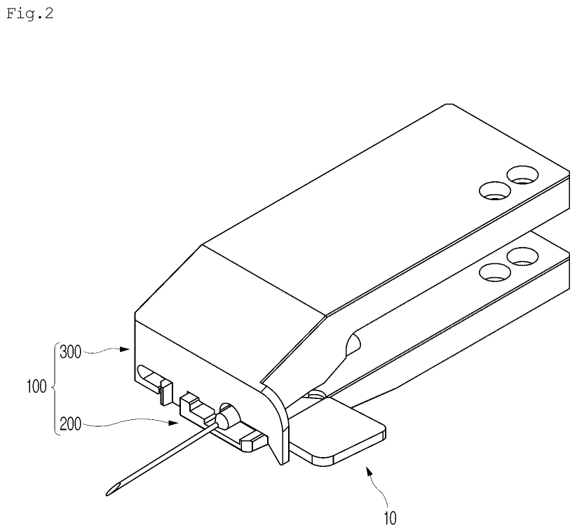

FIG. 2 is a perspective view showing a butterfly needle adapter device and the hold mode of a butterfly needle according to one embodiment of the present disclosure.

Referring to FIG. 2, a butterfly needle adapter device 100 according to one embodiment of the present disclosure may include a first clamp 200 and a second clamp 300. The first clamp 200 may be a member which supports the butterfly needle 10 from below and into which the butterfly needle 10 is inserted and seated. The second clamp 300 may be a member that supports the butterfly needle 10 to be inserted from the rear end side thereof to the front end side thereof and also guides the butterfly needle 10 through seating in an insert mode and prevents the butterfly needle 10 from being separated from the first clamp 200 in a hold mode.

Meanwhile, in one embodiment of the present disclosure, a state in which the butterfly needle 10 is completely disposed on the front end side of the first and second clamps 200 and 300 and is supported and fixed by the second clamp 300 may be referred to as a hold mode. In this case, the front end may be the front portion of the first clamp 200 and/or the second clamp 300, and may be a portion adjacent to the portion where the butterfly needle 10 is finally and completely disposed, that is, a portion adjacent to the portion that is disposed close to an invasion target during an invasion procedure.

Furthermore, in one embodiment of the present disclosure, a state in which the first and second clamps 200 and 300 are closed and/or a state in which the close arrangement of the first and second clamps 200 and 300 has been completed and thus the butterfly needle 10 is fixed may be referred to as a hold mode.

In one embodiment of the present disclosure, a state in which the butterfly needle 10 starts to be inserted or is being inserted from the rear end of the first and second clamps 200 and 300 to the front end thereof may be referred to as an insert mode. In this case, the rear end may be the rear portion of the first and second clamps 200 and 300, and may be a portion adjacent to a portion coupled with a driving unit 400 to be described later.

In addition, in one embodiment of the present disclosure, a state in which the first and second clamps 200 and 300 are open or a mode in which the first and second clamps 200 and 300 are changed from an open state to a closed state may be referred to as an insert mode. In the insert mode, the butterfly needle 10 may be inserted from the rear end side of the first and second clamps 200 and 300 to the front end side thereof. For example, the butterfly needle 10 may be moved by a user or may be electrically pushed forward by the driving unit 400 to be described later.

In one embodiment of the present disclosure, a state in which the butterfly needle 10 is separated from the first and second clamps 200 and 300 may be referred to as a release mode.

In addition, in one embodiment of the present disclosure, a mode in which the first and second clamps 200 and 300 are changed from a closed state to an open state may be referred to as a release mode.

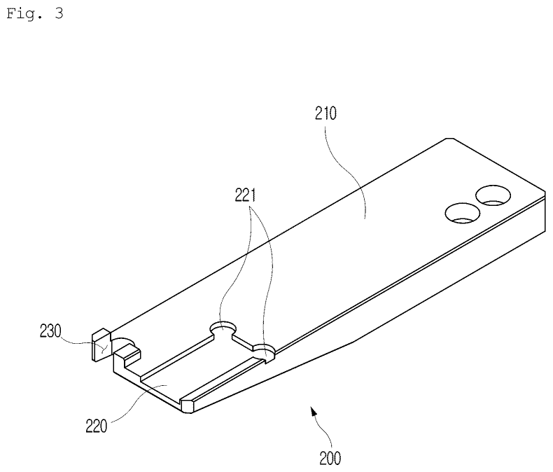

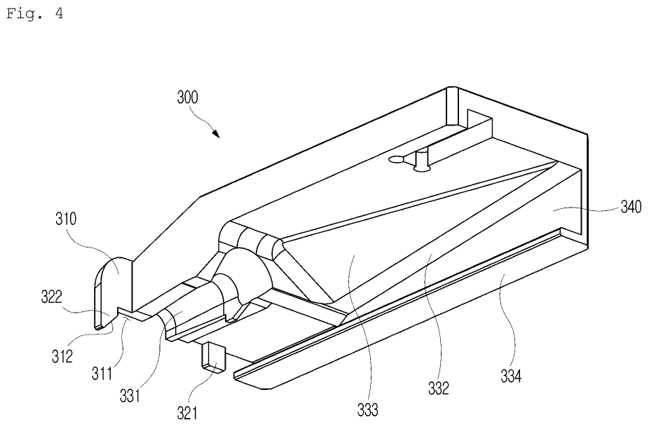

FIG. 3 is a diagram showing a first clamp according to one embodiment of the present disclosure.

Referring to FIG. 3, the first clamp 200 may include a flat support surface 210. The first clamp 200 may include a seat depression 220 that is formed concavely on the support surface 210 and allows the butterfly needle 10 to be seated therein in a hold mode. The seat depression 220 may be open in the forward direction from the first clamp 200. The seat depression 220 may be closed at the rear end thereof, so that it supports the rear end of the base support portions 142 of the butterfly needle 10 in the hold mode, thereby preventing the butterfly needle from being retreated. The seat depression 220 may include cylindrical recesses 221 on both sides of the rear end of the seat depression 220.

The first clamp 200 may include a reception portion 230 into which the protrusion 332 of the second clamp 300 to be described later is inserted in the hold mode. Due to the structure in which the protrusion 332 and the reception portion 230 are disposed, the second clamp 300 may be fixed not to move left and right relative to the first clamp 200 in the hold mode.

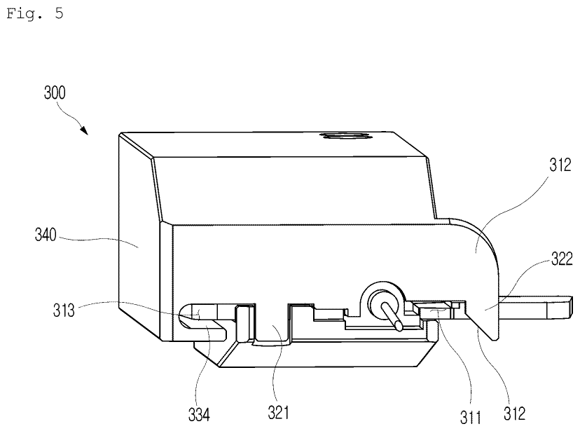

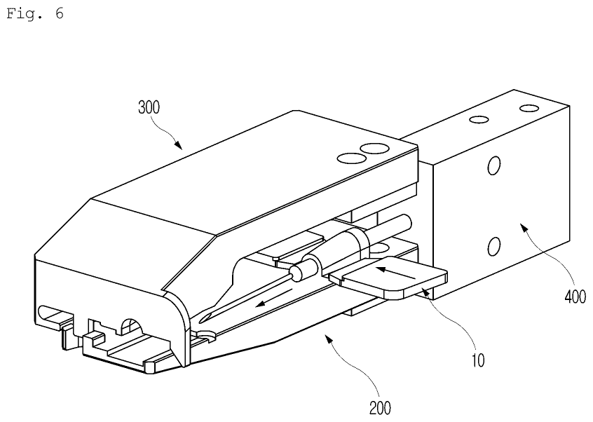

FIG. 4 is a diagram showing a second clamp according to one embodiment of the present disclosure. FIG. 5 is a diagram showing the front end of a butterfly needle adapter device according to one embodiment of the present disclosure.

Referring to FIGS. 4 and 5, the second clamp 300 may include a separation guide portion (not shown), a hold guide portion (not shown), an insertion guide portion (not shown), and a barrier wall 340. The separation guide portion may be a member that is intended to guide the butterfly needle 10 through separation in one lateral direction of the first and second clamps 200 and 300 in the release mode.

In this case, the one lateral direction may be the direction in which the open one side surface of the first and second clamps 200 and 300, via which the butterfly needle 10 is inserted or separated, faces. Furthermore, the one lateral direction may be the direction opposite to the direction in which the barrier wall 340 to be described later is disposed.

The separation guide portion may include a separation guide recess 311, a separation inclined portion 312, and a separation mark recess 313.

The separation guide recess 311 may be a portion that is disposed at the front end of the second clamp 300, is formed to extend toward one side of the second clamp 300, and guides the butterfly needle through separation in one lateral, horizontal direction. The separation guide recess 311 may be formed to extend in one lateral direction of the portion, which is open to allow the needle part 12 to be inserted, at the front end of the second clamp 300.

The separation inclined portion 312 may be a portion that extends on one side of the separation guide recess 311 and is inclined downward to one side.

The separation mark recess 313 may be a recess portion that is open at the front end of the second clamp 300 to check whether the butterfly needle 10 is seated or separated. A user may check whether the butterfly needle 10 is seated by checking the wing 16 of the butterfly needle 10 positioned on the straight movement guide portion 340 of the second clamp 300 via the separation mark recess 313.

The hold guide portion may be a portion that is disposed at the front end of the second clamp 300 and fixes the butterfly needle so that the butterfly needle does not move in the hold mode.

The hold guide portion may include a first protrusion 321 and a second protrusion 322 that protrude convexly downward. The first protrusion 321 may have a shape corresponding to the reception portion 230 of the first clamp 200 described above. In the hold mode, the first protrusion 321 may be inserted into the reception portion 230 and fix the second clamp 300 so that the second clamp 300 does not move left and right relative to the first clamp 200. The second protrusion 322 may be a portion where the above-described separation slope portion 312 is formed, and may be a portion that protrudes downward from the portion where the front end of the second clamp 300 extends in one lateral direction.

The forward movement of the butterfly needle 10 may be limited in such a manner that the wings 16 of the butterfly needle 10 collide with the first protrusion 321 and second protrusion 322 of the hold guide part while the butterfly needle 10 move forward in the insert mode.

The insertion guide portion may be a portion that guides the butterfly needle 10 through insertion in the insert mode.

The insertion guide portion may include a taper guide portion 331, a first inclined guide portion 332, a second inclined guide portion 333, and a straight movement guide portion 334.

The taper guide portion 331 is intended to support the needle connection support portion 141 described above in the hold mode, and may include a curved portion corresponding to the curved portion of the needle connection support portion 141. Furthermore, the taper guide portion 331 may be formed such that the portion thereof facing the needle connection support portion 141 in the hold mode is similar to the outer surface of the needle connection support portion 141.

The first inclined guide portion 332 may be a portion that is inclined downward from the rear end of the second clamp 300 toward the front end thereof. The first inclined guide portion 332 may be a portion that guides the butterfly needle 10 through insertion while supporting the butterfly needle 10 from above in the insert mode. For example, the first inclined guide portion 332 may guide the butterfly needle 10 through forward and downward movement while supporting a portion of the top surface of the right wing 16 of the butterfly needle 10 downward.

The second inclined guide portion 333 may be a portion that is inclined upward in one lateral direction while being inclined downward from the rear end side of the second clamp 300 toward the front end side thereof. The second inclined guide portion 333 may be a portion that guides the butterfly needle 10 through insertion while supporting the butterfly needle 10 in the downward direction and the other lateral direction opposite to the one lateral direction in the insert mode. For example, the second inclined guide portion 333 may guide the butterfly needle 10 through downward movement to the left side while supporting a portion of the top surface of the right wing 16 of the butterfly needle 10 downward to one side.

The straight movement guide portion 334 may be a portion that extends straight from the rear end of the second clamp 300 to the front end thereof. The straight movement guide portion 334 may be a portion that guides the butterfly needle 10 through straight insertion while supporting a portion of the bottom of the wing 16 of the butterfly needle 10. For example, the straight movement guide portion 334 may support a portion of the bottom of the right wing 16 of the butterfly needle 10 when the butterfly needle 10 is inserted, so that the right wing 16 can move forward along the straight movement guide portion 334.

The barrier wall 340 may be a portion that is formed to prevent the butterfly needle 10 from being separated in an unintended direction from the first and second clamps 200 and 300 during the process in which the butterfly needle 10 is inserted or separated. The barrier wall 340 may be formed on the side of the second clamp 300 opposite to the one side thereof. The lower end of the barrier wall 340 may be connected to the straight movement guide portion 334, and a portion of the barrier wall 340 may be connected to the first inclined guide portion 332.

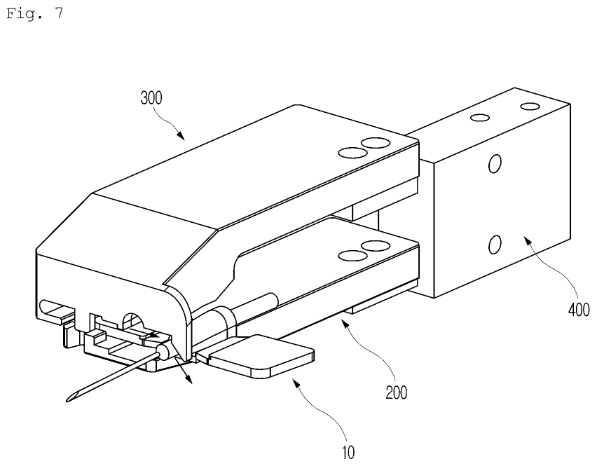

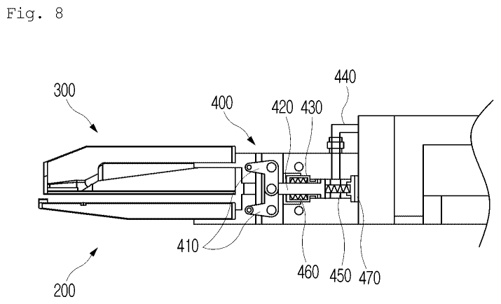

FIG. 6 is a diagram showing a butterfly needle adapter device including a driving unit according to one embodiment of the present disclosure. FIG. 7 is a diagram illustrating the operation of the driving unit in a hold mode according to one embodiment of the present disclosure. FIG. 8 is a diagram illustrating the operation of the driving unit in a release mode according to one embodiment of the present disclosure.

Referring to FIGS. 6 to 8, the butterfly needle adapter device 100 according to one embodiment of the present disclosure may further include a driving unit 400.

The driving unit 400 may serve to drive the first and second clamps 200 and 300 so that they can selectively approach each other and be separated from each other. Furthermore, the driving unit 400 may also serve to connect the first clamp 200, the second clamp 300, and the butterfly needle adapter device 100.

The driving unit 400 may include a pair of links 410, a piston 420, a cylinder 430, an actuator 440, and an elastic member 450.

First ends of the pair of links 410 may be coupled to the first and second clamps 200 and 300, respectively. Each of the links 410 may be formed in an inverted and reversed “L” shape. The bents of the links 410 may be pivotally coupled to the butterfly needle adapter device 100. The other ends of the links 410 may be coupled to the piston 420 to be described later. The pair of links 410 may be rotated in opposite rotational directions by the linear reciprocating movement of the piston 420.

The piston 420 may pivot the pair of links 410 in opposite directions while reciprocating linearly, and the front end of the piston 420 may be coupled to the other ends of the pair of links 410. Accordingly, when the piston 420 moves in the direction away from the first and second clamps 200 and 300, the pair of links 410 are rotated in the directions in which the first and second clamps 200 and 300 approach each other, so that the first and second clamps 200 and 300 can approach each other and fix the butterfly needle 10 seated on the wing support surface 210. In this case, the piston 420 may also be moved in the direction away from the first and second clamps 200 and 300 by an actuator 440 to be described later.

In this case, a pneumatic motor, a hydraulic motor, an electromagnet, a vacuum pump, or a piezoelectric element may be used as the actuator 440. However, the actuator 440 is not limited thereto.

The piston 420 may be moved in the direction toward the first and second clamps 200 and 300 by the elastic member 450 to be described later, and may be returned to its original position. In this manner, when the piston 420 is moved in the direction toward the first and second clamps 200 and 300, the pair of links 410 are rotated in the directions in which they are separated from each other, so that the first and second clamps 200 and 300 can be separated from each other and release the fixation of the butterfly needle 10.

The cylinder 430 is provided inside the driving unit 400. The piston 420 may be provided inside the cylinder 430 so that it can be reciprocated therein.

When power is applied, the actuator 440 may drive the piston 420 so that the pair of links 410 can approach each other. Furthermore, when power is removed, the actuator 440 may release the driving of the piston 420. More specifically, when the power of the actuator 440 is applied, the piston 420 is moved in the direction away from the first and second clamps 200 and 300, so that the pair of links 410 can approach each other and the first and second clamps 200 and 300 can also approach each other and fix the butterfly needle 10. When the power of the actuator 440 is removed, the piston 420 is moved in the direction toward the first and second clamps 200 and 300 by the elastic force of the elastic member 450 to be described later, so that the pair of links 410 can be separated from each other and the first and second clamps 200 and 300 can also are separated from each other and release the fixation of the butterfly needle 10.

In one embodiment of the present disclosure, the actuator 440 may move the piston 420 in the direction away from the first and second clamps 200 and 300.

Only when the power of the actuator 440 is applied do the first and second clamps 200 and 300 approach each other and fix the butterfly needle 10, so that the time for which power is applied to the actuator 440 can be minimized.

The elastic member 450 may be provided in the cylinder 430 and generate elastic force in the piston 420 so that the pair of links 410 can be separated from each other. The elastic member 450 generates elastic force that moves the piston 420 in the direction toward the first and second clamps 200 and 300. The elastic member 450 may be formed in an operation space to be described later.

In one embodiment of the present disclosure, the cylinder 430 may be provided with a sealing member 460 and a gasket 470.

The sealing member 460 may be provided at one end of the cylinder 430 adjacent to the first and second clamps 200 and 300, and may come into close contact with the outer surface of the piston 420. This sealing member 460 serves to seal the piston 420 and the cylinder 430.

For example, the sealing member 460 is not particularly limited, but an oil seal, a rubber packing, or the like may be used as the sealing member 460.

The gasket 470 covers the other end of the cylinder 430 that is relatively not adjacent to the first and second clamps 200 and 300. The gasket 470 may be provided at a distance from the piston 420. In this case, an operation space may be formed between the gasket 470 and the piston 420. The elastic member 450 configured to elastically support the gasket 470 and the piston 420 may be provided in this operation space.

When a negative pressure is formed in the operation space by the driving of the actuator 440, the piston 420 may be moved in the direction away from the first and second clamps 200 and 300. Furthermore, when the negative pressure formed in the operation space is released by the stopping of the actuator 440, the piston 420 may be moved in the direction toward the first and second clamps 200 and 300 by the elastic force generated from the elastic member 450.

Meanwhile, the axis from which the driving unit 400 extends and the axis from which the needle part 12 of the butterfly needle 10 extends may be formed on the same axis. Accordingly, the angle formed between the driving unit 400 and the skin of a blood collection target may be the same as the angle formed between the needle part 12 of the butterfly needle 10 and the skin of the blood collection target. In this case, a transfer device may transfer the driving unit 400 so that the needle part 12 of the butterfly needle 10 fixed to the first and second clamps 200 and 300 provided in the driving unit 400 is made to invade or is separated from the skin of a blood collection target along a set movement path and a set angle.

In addition, the driving unit 400 may be provided with a control unit (not shown) that checks the state of the needle part 12 of the butterfly needle 10, applied by the transfer device, via an image.

In this case, the state of the needle part 12 of the butterfly needle 10 checked by the control unit may be the state in which the butterfly needle 10 and the first and second clamps 200 and 300 are fixed to each other, the moving state of the needle part 12, or the angle formed between the needle part 12 and the skin.

While the needle part 12 of the butterfly needle 10 is made to invade or is separated from the skin of a blood collection target along a set movement path and a set angle by the transfer device, the control unit extracts the outline of the needle part 12 from the image based on deep learning, and then reads the consistency between an actual movement path based the three-axis coordinate positions of the needle part 12 and the set movement path and the consistency between an actual angle formed by the needle part 12 and the skin of a blood collection target and the set angle.

Thereafter, when the fixation of the butterfly needle 10 to the first and second clamps 200 and 300 is released, when an actual movement path based on the three-axis coordinate positions of the needle part 12 and the set movement path are not consistent with each other, or when an actual angle formed between the needle part 12 and the skin of a blood collection target and the set angle are not consistent with each other, the control unit may control the driving unit 400 and the transfer device so that the butterfly needle 10 is fixed to the first and second clamps 200 and 300 or the three-axis coordinate position of the needle part 12 is corrected. By the control of this control unit, the transfer device may move the needle part 12 so that the actual movement path based on the three-axis coordinate positions of the needle part 12 of the butterfly needle 10 and the set movement path are consistent with each other and the actual angle formed between the needle part 12 and the skin of the blood collection target and the set angle are consistent with each other.

In one embodiment of the present disclosure, the control unit may obtain the actual angle between the needle part 12 of the butterfly needle 10 and the skin of the blood collection target based on an ultrasonic image from an ultrasonic probe, and may obtain the actual movement path of the needle part 12 via a magnetic sensor, an ultraviolet camera, or an image camera.

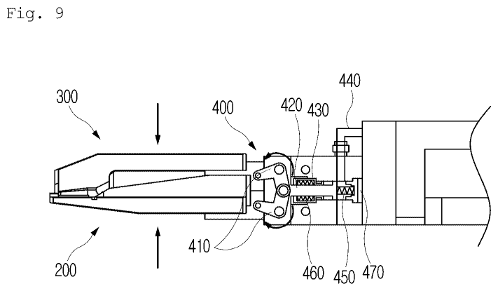

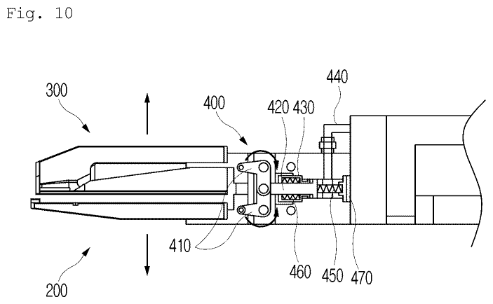

FIG. 9 is a diagram showing an insert mode for a butterfly needle according to one embodiment of the present disclosure. FIG. 10 is a diagram showing a release mode for a butterfly needle according to one embodiment of the present disclosure.

The operation of a butterfly needle adapter device according to one embodiment of the present disclosure will be described below.

Referring to FIGS. 9 and 10, first, any one of the pair of wings 16 of the butterfly needle 10 may be seated on the straight movement guide portion 334 of the insertion guide portion of the second clamp 300. In the insert mode, a user may easily seat one of the wings 16 of the butterfly needle 10 on the support surface 210 by moving the one wing 16 of the butterfly needle 10 along the insertion guide portion while holding the other wing 16 of the butterfly needle 10.

Next, in the hold mode, the piston 420 may be moved in the direction away from the first and second clamps 200 and 300 by the actuation of the actuator 440. As a result, the pair of links 410 are rotated in the directions in which they approach each other, so that the first and second clamps 200 and 300 can approach each other and fix the butterfly needle 10 seated on the taper guide portion 331.

More specifically, the needle support part 14 of the butterfly needle 10 and the tube 18 of the butterfly needle 10 are seated in the taper guide portion 331, the base support portion 142 is seated in the seat depression 220, and the rear end surface of the base support portion 142 is caught on the rear end surface of the seat depression. Accordingly, the rearward movement of the butterfly needle 10 is prevented, so that the hold mode can be completed.

Thereafter, blood collection from a blood collection target may be performed through the needle part 12 of the butterfly needle 10.

Thereafter, in the release mode, the actuator 440 is stopped, and the piston 420 is moved in the direction toward the first and second clamps 200 and 300 by the elastic force generated by the elastic member 450. As a result, the pair of links 410 are rotated in the directions in which they are separated from each other, so that the first and second clamps 200 and 300 can be separated from each other and release the fixation of the butterfly needle 10.

Finally, the one wing 16 of the butterfly needle 10 seated on the support surface 210 of the first clamp 200 may be separated therefrom. In the release mode, the user may easily remove the butterfly needle 10 by gripping the other wing 16 of the butterfly needle 10, moving the butterfly needle along the separation guide recess 311 in one lateral direction, and then moving it downward to one side along the separation inclined portion 312.

The butterfly needle adapter device 100 according to one embodiment of the present disclosure enables the butterfly needle 10 to be easily inserted and separated into and from the butterfly needle adapter device 100 in order to efficiently perform automatic blood collection.

In addition, the butterfly needle adapter device 100 according to one embodiment of the present disclosure includes the separation guide portion, thereby providing an effect of intuitively and easily performing the separation or removal of the butterfly needle 10.

In addition, the butterfly needle adapter device 100 according to one embodiment of the present disclosure includes the separation guide portion so that the butterfly needle 10 is separated via a side of the butterfly needle adapter device 100 after blood collection, thereby providing an effect of preventing the butterfly needle adapter device 100 from being contaminated with contaminants attached thereto when the butterfly needle 10 is separated via the rear side of the butterfly needle adapter device 100 and also preventing secondary infection.

In addition, the butterfly needle adapter device 100 according to one embodiment of the present disclosure includes the hold guide portion, thereby enabling the butterfly needle to be fixed without moving during blood collection.

In addition, the butterfly needle adapter device 100 according to one embodiment of the present disclosure includes the insertion guide portion, thereby providing an effect of enabling the butterfly needle 10 to be easily inserted from the rear end of the first clamp 200 and the second clamp 300 to the front end thereof.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1. A butterfly needle adapter device for holding a butterfly needle, the butterfly needle adapter device comprising:
a second clamp configured such that the butterfly needle is inserted and seated thereinto; and
a first clamp configured to prevent the butterfly needle from being separated from the second clamp in a hold mode;
wherein the second clamp comprises a separation guide portion that guides the butterfly needle through separation via one side of the second clamp in a release mode.

2. The butterfly needle adapter device of claim 1, wherein in an insert mode, the butterfly needle is inserted from a rear end side of the second clamp to a front end side thereof.

3. The butterfly needle adapter device of claim 1, wherein the separation guide portion comprises a separation guide recess that is disposed at a front end of the second clamp and formed to extend toward one side of the second clamp.

4. The butterfly needle adapter device of claim 3, wherein the separation guide portion comprises a separation inclined portion that extends on one side of the separation guide recess and is inclined downward to one side.

5. The butterfly needle adapter device of claim 1, wherein:
the second clamp comprises a butterfly needle hold guide portion at a front end thereof; and
the hold guide portion comprises a first protrusion protruding convexly downward.

6. The butterfly needle adapter device of claim 5, wherein the hold guide portion comprises a second protrusion that extends in one lateral direction and protrudes convexly downward.

7. The butterfly needle adapter device of claim 1, wherein the second clamp comprises an insertion guide portion that guides the butterfly needle through insertion in an insert mode.

8. The butterfly needle adapter device of claim 6, wherein the insertion guide portion comprises a first inclined guide portion that is inclined downward from a rear end side of the second clamp toward a front end side thereof.

9. The butterfly needle adapter device of claim 6, wherein the insertion guide portion comprises a second inclined guide portion that is inclined downward from a rear end side of the second clamp toward a front end side thereof and is also inclined upward in the one lateral direction.

10. The butterfly needle adapter device of claim 6, wherein the insertion guide portion comprises a straight movement guide portion that extends straight from a rear end side of the second clamp toward a front end side thereof.

11. The butterfly needle adapter device of claim 1, wherein the first clamp comprises a seat depression in which the butterfly needle is seated in the hold mode.

12. The butterfly needle adapter device of claim 10, wherein the seat depression is open forward of the first clamp.

13. The butterfly needle adapter device of claim 10, wherein a rear end of the seat depression is closed and thus prevents the butterfly needle from retreating in the hold mode.

14. The butterfly needle adapter device of claim 5, wherein the first clamp comprises a reception portion into which the first protrusion is inserted in the hold mode.
